(19) United States
(12) Patent Application Publication
(10) Pub. No.: US 2014/0207190 A1
(43) Pub. Date: Jul. 24, 2014

(54) SPINAL THREE-DIMENSIONAL ORTHOPEDIC EQUIPMENT

(71) Applicants: JILIN ZHANG, Beijing City (CN); YI ZHANG, Beijing City (CN)

(72) Inventors: JILIN ZHANG, Beijing City (CN); YI ZHANG, Beijing City (CN)

(21) Appl. No.: 14/222,673

(22) Filed: Mar. 23, 2014

Related U.S. Application Data
(63) Continuation-in-part of application No. 13/000,000, filed on Dec. 17, 2010.
Application No. 13/000,000 is a continuation-in-part of application No. PCT/CN2008/071133, filed on May 29, 2008.

(51) Int. Cl. A61F 5/042

(52) U.S. Cl. CPC A61F 5/042
USPC 606/240

(57) ABSTRACT

A spinal three-dimensional orthopedic equipment comprises a frame, a front board member and a drive device for driving the front board member fixed on the frame, a back board member and a middle board member as well as their drive devices fixed on the frame, a head holding device fixed on the middle board member for holding the patient's head, and a drawing and holding device for drawing and holding the head and neck of the patient. The head holding device comprises a splint connected to the middle board member immovably, two sliding grooves are provided on the splint, and two head holding clips and a mandible tray connected to the sliding pieces mounted in the two sliding grooves respectively, which can slide along the sliding grooves. The spinal orthopedic equipment also comprises a front board member fast lifting device, and a drive device for controlling the lifting device.

### CROSS-REFERENCE TO RELATED APPLICATION(S)

The present application is a continuation-in-part of U.S. patent application Ser. No. 13/000,000 filed on Dec. 17, 2010, which is a 35 U.S.C. §371 National Phase conversion of International (PCT) Patent Application No. PCT/CN2008/071133, filed on May 29, 2008, the disclosure of which is incorporated by reference herein.

### TECHNICAL FIELD

The present invention relates to medical appliances and in particular to a medical appliance for remedying parenchyma trauma between spinal vertebras and for health care of spine.

### BACKGROUND OF THE INVENTION

Chinese patent (Patent No.: 97182091.0) disclosed a three-dimensional spine remedying apparatus for remedying the pathological changes caused by three-dimensional displacements between spinal vertebras merged with intervertebral parenchyma trauma. The three-dimensional spine remedying apparatus comprises a frame for mounting operating mechanisms and drive devices of the orthopedic equipment; a cephalothoracic board, which is fixed on said frame, for supporting and securing the upper torso of the patient, and capable of moving horizontally along the longitudinal axle (X) of the equipment; and a device for driving said cephalothoracic board; a hip-leg board, which is fixed on said frame, for supporting and securing the lower torso of the patient, and capable of rotating around the lateral axle (Y) and the longitudinal axle (X) of said apparatus; a device for rotating said hip-leg board around the axle Y; and a device for rotating said hip-leg board around the axle X; and an electric control system for controlling the moving speed and distance of foregoing mechanisms; a hip board fixed on said hip-leg board, which is capable of rotating around the vertical axle (Z) of said equipment and a device for driving said hip board. The spinal three-dimensional orthopedic equipment of the prior art can generate good treatment effect for such diseases as lumbar disc protrusion, thoracic vertebra/lumbar vertebra joint disorder sickness and lumbar muscle strain. This patent document also mentioned that the three-dimensional spine remedying apparatus can be used to treat cervical spondylosis. However, in the process of realizing the present invention the inventor discovered that, the three-dimensional spine remedying apparatus features no obvious difference from the traditional drawing method in the treatment of cervical spondylosis, and fails to stably fix the patient's head and achieve the location and three-dimensional coordinated actions of head and neck, leading to long treatment time and dissatisfactory treatment effect. For the treatment of cervical spondylosis, one of the most effective methods is to apply mechanical force to realize the fast movement between vertebras in the three-dimensional directions, correct the vertebrae malposition or misplacement and to release the intervertebral strained relation, so that the vertebras can return or approach their natural state. However, the spinal three-dimensional orthopedic equipment of the prior art fails to provide such movement. In the process of remedying parenchyma trauma between spinal vertebras by using said spinal three-dimensional orthopedic equipment, its drive devices can realize three-dimensional rotation actions, the drawing action in longitudinal direction as well as the action of transverse shearing, but does not provide the action of up-and-down shearing that can produce obvious effect in treatment. In addition, it lacks of squeezing action along longitudinal axis of the body when being used for health care of spinal. Only by means of such action of up-and-down shearing, it is feasible to achieve the three-dimensional ridge correction in real significance.

### SUMMARY OF THE INVENTION

It is the technical object of the present invention to provide a new omnibearing spinal three-dimensional orthopedic equipment for remedying the pathological changes caused by three-dimensional displacements between spinal vertebras more effectively and used for the purpose of health care for the spine, so as to overcome the deficiency of the prior art.

The following technical solutions are adopted to achieve the objective of the present invention and to address its technical issues. A spinal three-dimensional orthopedic equipment comprises a frame, a front board member and a drive device for driving the front board member fixed on the frame, a back board member and a drive device for driving the back board member fixed on the frame, and a middle board member and a drive device for driving the middle board member fixed on the frame. The spinal three-dimensional orthopedic equipment further comprises a head holding device movably placed on the middle board member for holding patient's head, and a drawing and holding device connected to the head and neck; said front board member makes level impact movement quickly towards positive and negative direction in certain distance along the longitudinal axle of the spinal orthopedic equipment, said back board member quickly turns around the transverse axle and longitudinal axle of the spinal orthopedic equipment, and said middle board quickly turns in perpendicular to the back board member and around the spinal orthopedic equipment which are completed synchronously within preset time.

Preferably, said head holding device further comprises a splint movably connected to said middle board member, a plurality of sliding grooves provided on the splint for sliding pieces sliding therein, two head holding clips each of which is connected to the sliding pieces via a rotating shaft so as to rotate around the rotating shaft relative to the splint, and a mandible tray for receive patient's mandible connected to the sliding pieces next to and space from the two head holding clips.

Preferably, said drawing and holding device further comprises a longitudinal drawing belt and a holding cap; said holding cap is fixed onto the head of the patient; one end of said longitudinal drawing belt is connected to the holding cap, and the other end of said longitudinal drawing belt is connected to said back board member.

Preferably, the spinal three-dimensional orthopedic equipment further comprises a fast lifting device mounted under said front board member, and a driving device for controlling the fast lifting device; the specific structure of the fast lifting device comprises a hinged shaft transversely mounted between underneath the splint for support of said front board member and said frame, the hinged shaft is rotated by the driving device for controlling said fast lifting device, and one end of said front board member next to the middle board member is capable of lifting or falling rapidly in a short distance.

Preferably, said driving device for driving said fast lifting device is driven by torque motor.

Preferably, the spinal three-dimensional orthopedic equipment further comprises a backup board, the backup board and the front board member jointly form a drawing-pulling structure or a movable structure so that the backup board is capable of being attached to and detached from the front board member; the backup board forming the drawing-pulling structure is provided in the sliding groove of the front board member therein and capable of be moved in push-and-pull way along the sliding groove; one end of the backup board form the movable structure is hinged jointly with the front end of the front board member.

Preferably, the spinal three-dimensional orthopedic equipment further comprises two grooves placed on the front board member and on positions of the front board member corresponding to two breasts of a patient taking a prone position for treatment.

Preferably, a pressure sensor is provided on the front board member.

Preferably, the spinal three-dimensional orthopedic equipment further comprises a control computer connected to the pressure sensor.

Preferably, the each holding clip comprises an ear hole used to accommodate patient's ears.

As compared with the prior art, the present invention has obvious advantages and beneficial effects. By adopting the spinal three-dimensional orthopedic equipment of the present invention, it is feasible to achieve the three-dimensional ridge correction in real significance, automatically treat the patient with parenchyma trauma between body spinal vertebras or rehabilitation of human body.

Said description merely provides the overview of the technical solution of the present invention. The present invention is described herein based on preferred embodiments in combination with the attached drawings, so that the technical means of the present invention can be understood more clearly and implemented in accordance with the content of the product description, and the objectives, characterizes and advantages of the present invention are easier for understanding.

### BRIEF DESCRIPTION OF THE FIGURES

Further detailed description of the present invention is provided herein in combination with the attached figures.

FIG. 1 is the structural diagram of the spinal three-dimensional orthopedic equipment of the present invention.

FIG. 2 is the top view illustrating the head holding device of the spinal three-dimensional orthopedic equipment of the present invention.

FIG. 3 is the schematic diagram illustrating the operating state of the head holding device of the spinal three-dimensional orthopedic equipment of the present invention.

FIG. 4 is the top view illustrating the front board member of the spinal three-dimensional orthopedic equipment of the present invention.

FIG. 5 is the three-dimensional diagram illustrating holding cap of the spinal three-dimensional orthopedic equipment of the present invention.

### DETAILED DESCRIPTION OF THE PREFERRED EMBODIMENT

To further elaborate the technical means adopted to achieve the objective and effect of the present invention, the spinal three-dimensional orthopedic equipment and its specific structure, application method and effect of the present invention is further described in detail in combination with the attached drawings and preferred embodiments.

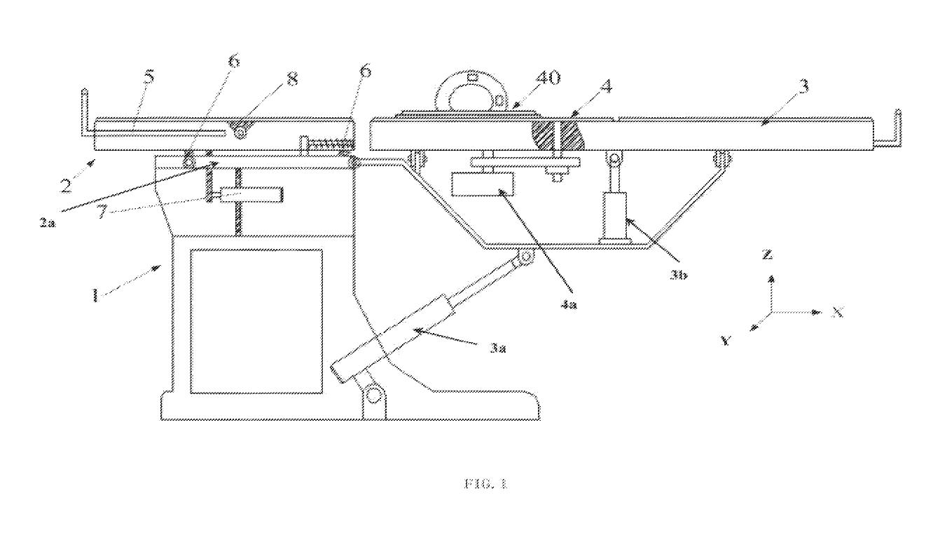

FIG. 1 is the structural diagram of the spinal three-dimensional orthopedic equipment of the present invention. The spinal three-dimensional orthopedic equipment comprises a frame 1 for mounting operating mechanisms and drive devices of the orthopedic equipment; a front board member 2, which is mounted on the frame 1, for supporting and securing the upper torso of the patient when the patient is in a first treatment position on the orthopedic equipment (Referring to FIG. 3), and capable of making level impact movement quickly towards positive and negative direction in certain distance along the longitudinal axle (namely, X direction) of the spinal orthopedic equipment; a drive device 2a for driving the front board member 2 to make fast movement; a back board member 3, which is mounted on the frame 1, for supporting the lower torso of the patient in remedying the parenchyma trauma between thoracic/lumbar vertebras when the patient is in a second treatment position on the orthopedic equipment (Details regarding the second treatment position referring to previously mentioned Chinese Patent No. 97182091.0), and capable of rotating around the lateral axle (namely, Y direction) of the orthopedic equipment; a drive device 3a for driving the back board member 3 to rotate around Y-direction axle; a drive device 3b for driving the back board member 3 to rotate around X-direction axle; a middle board member 4, which is mounted on the back board member 3, capable of rotating rapidly around the axle being perpendicular to the back board surface of the orthopedic equipment (namely Z-direction axle), for implementing transverse shearing between the vertebras with pathologic changes of the patient; a drive device 4a for driving the middle board member 4 to rotate around Z-direction axle; as well as an informational and electric control system for controlling the movement velocities and distances of operating mechanisms (not shown in the FIGS.). Under the control of informational and electrical control system, the spinal three-dimensional orthopedic equipment of the present invention could make the front board member 2 that acts on pathological inter vertebral impact quickly, the back board member 3 rotate around X-axle quickly, and the middle board member 4 twist fast around Z-axle which are completed synchronously within 0.3 second; the impact stroke of the front board member 2 is adjustable within the scope of 10 mm-90 mm according to specific lesion location, lesion degree, the patient' height and body weight, and the whole impact process is controlled to be completed within 0.25 second. The informational and technical control could make fast impact and pulling on vertebra with pathological changes that is different from available pulling. Its quick action could help eliminate resist from myotatic reflex of muscles, avoid unnecessary tissue injury, make vertebrae clearance increasing instantly, and release adhesion of nerves surrounding inter vertebrae, all of which is favorable for improvement of lesion spinal nerve; the fast dynamic load is within the affordable elastic limit of parenchyma of the body and will neither cause damage to normal tissues, nor cause pain. The back board member 3 rotates around X-axle quickly adjustably within ±40 degree, the rapidly rotating force could act on lesion inner vertebrae, rectify dislocation of inner vertebrae and help release adhesion of parenchyma of inter vertebrae; the middle board member 4 fast twist around Z-axle adjustably within ±20 degree, and such movement have transverse shear action on lesion vertebrae and could rectify dislocation and release adhesion; all these actions synchronize their movements for completion instantly, which help restore structure of spinal vertebrae to natural state, release the strained relations of surrounding lesion nerves, and is favorable for restore and cure damaged nerves. In the treatment of cervical spondylosis, a head holding device 40 used for holding the head of the patient in the first treatment position and a drawing and holding device connected to the head and neck are removable mounted on the middle board member 4, so as to treat the cervical spondylosis of the patient.

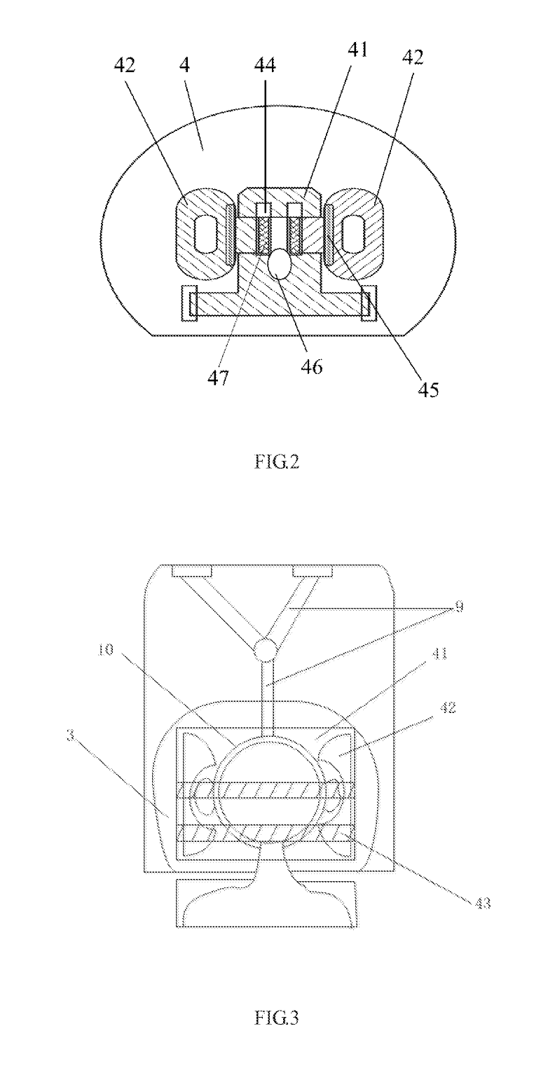

As shown in FIG. 2 and FIG. 5, the head holding device 40 comprises a splint 41, holding clips 42 and fastening strap 43. The splint 41 is tabular, below which a leg-shaped structure is provided, its leg is inserted and fixed to the strap hole of the middle board member 4 and can make synchronized movement along with the movement of the middle board member 4; the fastening strap 43 and its quantitative tensioning device have been described in the aforementioned Chinese Patent (Patent No. 97182091.0). Two sliding grooves 44 are provided on the splint 41, and two head holding clips 42 are provided to hold the head of the patient, each holding clip has a rotating shaft 45; the rotating shaft 45 is connected with to the sliding pieces 47 mounted in the sliding groove 44 and a mandible tray 46, so that the holding clips in together with the mandible tray 46 can make longitudinal sliding motion in the sliding groove 44. In addition, two holding clips also make open and close movement around the rotating shaft 45. Ear holes are provided on the holding clips 42, which are used to accommodate the ears in securing the head of the patient to prevent the ears of the patient from injury due to extrusion. At the same time, the ear holes can also guarantee satisfactory hearing acuity when the patient is accepting treatment. The fastening strap 43 is mounted on the middle board member 33, and is used for fastening the pelvis in remedying the disease between lumbar vertebras and for securing the head of the patient placed on the splint 41 in the treatment of cervical spondylosis. The method is to use the fastening strap 43 to wrap two holding clips and allow them to secure the head of the patient, so that the head of the patient can be stably secured on the head holding device 40. It is preferred that the fastening strap 43 is a device with adjustable tensioning force. The drawing and holding device comprises a longitudinal drawing belt 9 and a holding cap 10, and is used to draw and hold the head and neck of the patient. The holding cap 10 is fixed onto the head of the patient. One end of the longitudinal drawing belt 9 is connected to the holding cap 10, and the other end of the longitudinal drawing belt 9 is connected to the back board member 3.

FIG. 3 is the schematic diagram when the head of the patient is secured on the head holding device 40 for treatment. On the head holding device 40, under the fixation effect of the longitudinal drawing belt 9, the holding cap 10 and the head holding device 40, the head of the patient can stably move in together with the middle board member 4 and can accept the three-dimensional coordinated actions of three quantitative boards of the spinal three-dimensional orthopedic equipment and complete instant impact and pulling, twisting and shearing on lesion intervertebral of cervical vertebrae, put the location of inter vertebrae straight, smooth the disordered relationship between vertebrae, release abnormal tension of nerves and vein of inter vertebrae and make the vertebrae restore to or develop towards natural state, so as to achieve cure.

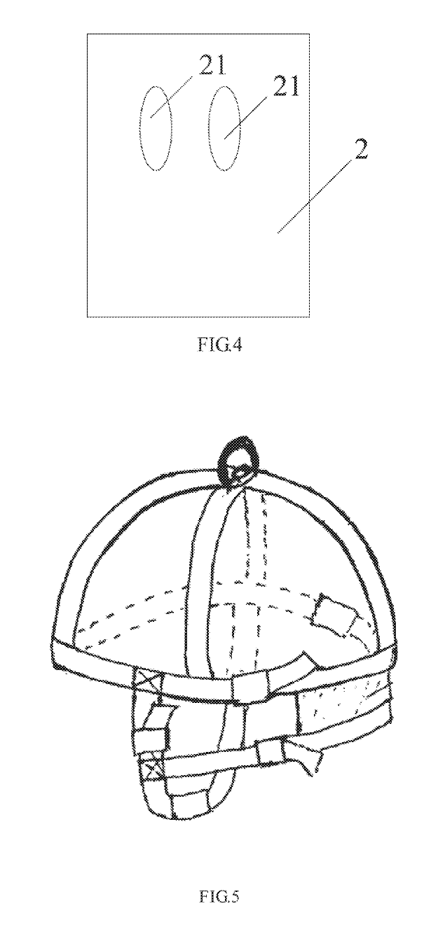

For the treatment of disease between vertebras, the patient is required to take the prone position on the front board. In this case, the chest and breasts of a female patient will be subject to extrusion, which may cause adverse consequence. As shown in FIG. 4, two grooves 21 are set on the front board 2 of the spinal three-dimensional orthopedic equipment, and soft elastic material is filled in the grooves 21, or, on the positions in the front board member where two breasts are located when the patient is taking the prone position for treatment, a sag lifting device and its driving mechanism are provided to accommodate the chest and breasts of the patient in the process of treatment, so as to improve the comfortableness of the spinal three-dimensional orthopedic equipment in treatment.

As shown in FIG. 1, for the spinal three-dimensional orthopedic equipment of the prior art, since its front board member is mainly used for supporting the upper torso of the patient in the treatment of lumbar intervertebral disease, its length is limited. In the treatment of cervical spondylosis, the front board member 2 is used for supporting the whole torso below the head of patient. Therefore, the spinal three-dimensional orthopedic equipment of the present invention further comprises a backup board 5, the backup board 5 and the front board member 2 jointly form a drawing-pulling structure or a movable structure so that the backup board 5 is capable of being attached to and detached from the front board member 2. The backup board forming the drawing-pulling structure is provided in the sliding groove of the front board member therein and capable of be moved in push-and-pull way along the sliding groove. In the process of treatment, the backup board 5 is drawn out. Upon completion of treatment, the backup board 5 can be pushed into the front board member 2. One end of the backup board 5 form the movable structure is hinged jointly with the front end of the front board member 2, so as to support the leg and foot of the patient, level the backup board 5 and the front board member 2 and hung on a supporting hook; when take down the supporting hook and make the backup board verticality hung, it can be used for treating the patient with thoracic vertebra/lumbar vertebra disease.

In order to treat the disease between vertebras more effectively, it is necessary to move the prone patient' vertebras with pathologic changes up and down in the process of treatment, so as to generate infinitesimal displacement of them in vertical direction. To realize said objective, a fast lifting device 6 and a drive device 7 for controlling the fast lifting device 6 are mounted below the front board member, the fast lifting device 7 can be designed as a cam or an eccentric cam set below the front board member as well as its drive device. The specific structure of the fast lifting device 6 comprises a hinged shaft transversely mounted between underneath the splint for support of said front board member and said frame, the hinged shaft is rotated by the driving device for controlling said fast lifting device, and one end of said front board member next to the middle board member is capable of lifting or falling rapidly in a short distance, so as to realize back and forth shearing effect on lesion inner vertebrae. This function could also be realized by the cam or eccentric wheel and their driving devices mounting under the front board member.

Preferably, a pressure sensor 8 is provided on the front board member to measure the weight endured on the front board member, so as to acquire the body weight data of the patient and provide data on more aspects for treatment. The pressure sensor is connected with a computer and thus can be used as a basis for automatic setup of treatment parameters, and can also make counting for the treated patients. When the pressure sensor fails to detect pressure, it is believed that there is no patient, and then the action is trial idle operation of the spinal three-dimensional orthopedic equipment.

The technical characteristics that are not described in detail in this application can be found in Chinese Patent (Patent No.: 97182091.0), and the corresponding technical effect can be achieved. Therefore, unnecessary details of the corresponding content will be no longer provided herein.

The spinal three-dimensional orthopedic equipment of the present invention is an automatable machine informational and technically controlled that could have its impact stroke, angled number, angle of swing and torsion angle and other parameters set according to lesion location, lesion degree, the patient' height and body weight and other specific circumstances, and could rectify lesion vertebrae and spine in three-dimensional way within the affordable safety limit of human body. It can not only effectively remedy the disease between thoracic vertebras/lumber vertebras, but also can effectively treat cervical spondylosis and used for health protection of spinal column. Furthermore, by means of the pressure sensor, it can automatically judge whether there is a patient who is accepting treatment. Since a front board member fast lifting device is added, it is feasible to generate better treatment effect on the disease between vertebras.

The preferred embodiments of the present invention have been disclosed as above, but are not used to limit the present invention in any form. Those skilled in the art may make some changes or modifications to achieve equivalent embodiments by using the methods and technical content disclosed above. However, any simple modification, equivalent change and modification made to said embodiments based on the technical essence of the present invention without deviation from the technical solution of the present invention still falls within the claims of the present invention.

What is claimed is:

1. A spinal three-dimensional orthopedic equipment comprises a frame, a front board member and a drive device for driving the front board member fixed on the frame, a back board member and a drive device for driving the back board member fixed on the frame, and a middle board member and a drive device for driving the middle board member fixed on the frame, wherein the spinal three-dimensional orthopedic equipment further comprises a head holding device movably placed on the middle board member for holding patient's head, and a drawing and holding device connected to the head and neck; said front board member makes level impact movement quickly towards positive and negative direction in certain distance along the longitudinal axle of the spinal orthopedic equipment, said back board member quickly turns around the transverse axle and longitudinal axle of the spinal orthopedic equipment, and said middle board quickly turns in perpendicular to the back board member and around the spinal orthopedic equipment which are completed synchronously within preset time.

2. The spinal three-dimensional orthopedic equipment of claim 1, wherein said head holding device further comprises a splint movably connected to said middle board member, a plurality of sliding grooves provided on the splint for sliding pieces sliding therein, two head holding clips each of which is connected to the sliding pieces via a rotating shaft so as to rotate around the rotating shaft relative to the splint, and a mandible tray for receive patient's mandible connected to the sliding pieces next to and space from the two head holding clips.

3. The spinal three-dimensional orthopedic equipment of claim 1, wherein said drawing and holding device further comprises a longitudinal drawing belt and a holding cap; said holding cap is fixed onto the head of the patient; one end of said longitudinal drawing belt is connected to the holding cap, and the other end of said longitudinal drawing belt is connected to said back board member.

4. The spinal three-dimensional orthopedic equipment of claim 1, wherein the spinal three-dimensional orthopedic equipment further comprises a fast lifting device mounted under said front board member, and a driving device for controlling the fast lifting device; the specific structure of the fast lifting device comprises a hinged shaft transversely mounted between underneath the splint for support of said front board member and said frame, the hinged shaft is rotated by the driving device for controlling said fast lifting device, and one end of said front board member next to the middle board member is capable of lifting or falling rapidly in a short distance.

5. The spinal three-dimensional orthopedic equipment of claim 4, wherein said driving device for driving said fast lifting device is driven by torque motor.

6. The spinal three-dimensional orthopedic equipment of claim 1, wherein the spinal three-dimensional orthopedic equipment further comprises a backup board, the backup board and the front board member jointly form a drawing-pulling structure or a movable structure so that the backup board is capable of being attached to and detached from the front board member; the backup board forming the drawing-pulling structure is provided in the sliding groove of the front board member therein and capable of be moved in push-and-pull way along the sliding groove; one end of the backup board form the movable structure is hinged jointly with the front end of the front board member.

7. The spinal three-dimensional orthopedic equipment of claim 1, wherein the spinal three-dimensional orthopedic equipment further comprises two grooves placed on the front board member and on positions of the front board member corresponding to two breasts of a patient taking a prone position for treatment.

8. The spinal three-dimensional orthopedic equipment of claim 1, wherein a pressure sensor is provided on the front board member.

9. The spinal three-dimensional orthopedic equipment of claim 8, wherein the spinal three-dimensional orthopedic equipment further comprises a control computer connected to the pressure sensor.

10. The spinal three-dimensional orthopedic equipment of claim 1, wherein the each holding clip comprises an ear hole used to accommodate patient's ears.
